(19) United States
(12) Patent Application Publication
(10) Pub. No.: US 2019/0127691 A1
(43) Pub. Date: May 2, 2019

(54) Metabolic Pressure for Stem Cell Differentiation and Purification

(71) Applicant: The Charles Stark Draper Laboratory, Inc., Cambridge, MA (US)

(72) Inventors: Timothy Petrie, Quincy, MA (US); Jonathan R. Coppeta, Windham, NH (US); Dorit Berlin, Lexington, MA (US)

(21) Appl. No.: 16/172,364

(22) Filed: Oct. 26, 2018

Related U.S. Application Data
(60) Provisional application No. 62/577,247, filed on Oct. 26, 2017.

(51) Int. Cl. C12N 5/071; C12N 5/074

(52) U.S. Cl. CPC C12N 5/067; C12N 5/0696; C12N 2501/06; C12N 2506/45; C12N 2500/34

(57) ABSTRACT

Described herein are cell culture methods of producing hepatocytes, or mature, highly functional hepatocyte-like cells in vitro; cell culture media suitable for use in these methods; functional hepatocytes, or mature, highly functional hepatocyte-like cells produced by these methods; and cell compositions comprising hepatocytes, or mature, highly functional hepatocyte-like cells produced by these methods.

### RELATED APPLICATIONS

This application claims the benefit under 35 USC 119(e) of U.S. Provisional Application No. 62/577,247, filed on Oct. 26, 2017, which is incorporated herein by reference in its entirety.

### BACKGROUND OF THE INVENTION

Induced pluripotent stem cells (iPSCs) are mature/adult cells that have been genetically reprogrammed to an embryonic stem cell-like state by being forced to express genes and factors important for maintaining the defining properties of embryonic stem cells. IPSCs are useful tools for drug development and modeling of diseases, and for transplantation therapies to develop compatible tissue matched to the cell donor to avoid rejection by the immune system. (See, for example, www.stemcells.nih.gov/info/basics).

The liver is an essential organ of the mammalian species and its specialized functions include protein synthesis, glucose metabolism, carbohydrate metabolism, lipid metabolism and detoxification of compounds such as, for example, prescription drugs. The majority of parenchymal cells are hepatocytes. Isolated hepatocytes can be cultured in primary cell culture for use in therapeutic and pharmacological studies, and for potential transplantation. But using primary cell culture as a source of hepatocytes is insufficient to meet the great demand for these cells.

Some in vitro pluripotent stem cell differentiation protocols have been reported, but these protocols, whether using embryonic stem cells, or induced pluripotent stem cells, have produced hepatocyte-like cells lacking the functionality of mature hepatocytes. Therefore, there is still a need for an efficient process for producing mature, highly functional hepatocytes from stem cell cultures (either embryonic or induced pluripotent stem cells) on a large enough scale to meet the demand for hepatocytes, or mature, highly functional hepatocyte-like cells.

### SUMMARY OF THE INVENTION

Small molecules, peptides, and proteins can selectively activate or inhibit specific cellular metabolic pathways used for cellular energy production, or for creating critical cellular substrates. Specific nutrients or energy substrates, such as fructose or galactose can also act as metabolic stressors to direct hepatocyte lineage specification. In particular, small molecules, peptides, proteins, nutrients, substrates as well as culture conditions such as pH, and atmospheric conditions (e.g., oxygen, nitrogen concentrations), can act as metabolic stress agents, or metabolic stressors. Activating or inhibiting these pathways in stem cells can promote differentiation down specific cell lineages while also selecting against cells that are incompetent or non-viable to adapt to these metabolic changes. Therefore, proper metabolic stress induced at either discrete time points during the culture process, or as constant stress, e.g., increasing metabolic stress during culture, can reasonably both direct differentiation and purify cellular populations.

Cells obtain their energy to function mainly through two encompassing energy processes, glycolysis and oxidative phosphorylation. Hepatocytes, the primary metabolically active liver cells in-vivo, perform a number of mostly liver-specific metabolic programs. These processes include:

Use of alternate substrates for glycolysis metabolism (galactose, fructose

Internal production of glucose (gluconeogenesis)

Phenylalanine hydroxylation (boosts oxidative phosphorylation)

Ornithine metabolism

By pressuring cells to utilize only these metabolically unique processes, it is reasonable to believe that a heterogeneous pluripotent cell derived population will be enriched with cells predisposed to producing energy (vital for viability) via these liver-specific programs, resulting in mature highly functioning hepatocytes, or mature highly functioning hepatocyte-like cells with a hepatocyte phenotype.

Several metabolic pathway modifications are described herein in the context of differentiating stem cells, and specifically, induced pluripotent stem cells (iPSC) into a more functional in vivo like hepatic phenotype. As used herein, the terms for induced pluripotent stem cells such as “iPSC”, “PS”, or “iPSCs” will encompass both the singular as well as the plural. The present invention encompasses these metabolic methods for producing hepatocytes, or mature highly functioning hepatocyte-like cells, from mammalian (e.g., human, mouse, rat, pig, etc.) induced pluripotent stem cells, and specifically from human induced pluripotent stem cells (hiPSC). Although iPSC are specifically described herein, it is reasonable to believe that such methods can also use mammalian embryonic stem cells, and specifically human embryonic stem cells.

Specifically encompassed by the present invention are methods of producing hepatocytes, or mature hepatocyte-like cells from induced pluripotent stem cells, comprising the steps of (or consisting essentially of the steps) culturing the induced pluripotent stern cells under conditions suitable for inducing metabolic stress, either continuously or at different; distinct stages of hepatic lineage specification (see e.g., FIG. A-B). Stage-dependent metabolic pressure induces the stem cells to differentiate into hepatocytes or mature highly functional hepatocyte-like cells, thereby producing hepatocytes, or mature highly functional hepatocyte-like cells or enriching a population of iPSCs with hepatocytes, or mature highly functional hepatocyte-like cells. In one embodiment of the present invention, the iPSCs are mammalian cells, and in particular, human iPSCs.

More specifically, the present invention encompasses methods wherein the metabolic stress condition (resulting from e.g., a single stressor such as a specific peptide, or a cocktail/combination of metabolic stressors) is continuous throughout the cell culture conditions. Alternatively, also encompassed are methods wherein the metabolic stress condition(s) is induced at specific times during cell culture at the hepatoblast lineage specification stage (e.g., specification, early maturation or late maturation steps). Such a stress condition can be introduced/induced during cell culture at the early/mid-phase of the hepatic lineage specification stage, or is induced during cell culture at the late-phase of the hepatic lineage specification stage.

Also encompassed by the present invention are cell culture medium/media comprising reagents or components suitable for differentiating induced pluripotent stem cells into hepatocytes, or mature, highly functional hepatocyte-like cells comprising glycolysis inhibitors. In one embodiment, the culture medium comprises one, or more, oxidative phosphorylation inhibitors such as rotenone, oligomycin, or rutamycin. In another embodiment, the culture medium comprises one, or more, metabolic substrates at concentrations suitable for inducing hepatic-specific metabolic processes for energy production such as galactose, fructose, or ornithine.

Another embodiment of the present invention encompasses isolated cells, or an isolated population of cells comprising, or enriched in, hepatocytes, or mature, highly functional hepatocyte-like cells produced by the methods described herein. More specifically, an isolated cell population, or a composition comprising a cell population of at least about 80% to about 90% or greater of hepatocytes; or least about 80% to about 90% or greater of a combination of hepatocytes and mature, highly functional hepatocyte-like cells; or least about 80% to about 90% or greater of mature, highly functional hepatocytes, is encompassed by the present invention.

Using the methods described herein, one can induce, i.e., direct, the differentiation of iPSC when iPSC are cultured under the appropriate conditions specified herein, resulting in a population of differentiated cells with the functional characteristics of mature human hepatocytes. More specifically, culturing the iPSC under the conditions described herein results in the production (i.e., augment iPS-derivation, differentiation of, or selection of) a population of cells substantially enriched in hepatocytes, or hepatocyte-like cells.

As used herein, the terms “hepatocytes” or “mature highly functioning hepatocyte-like cells” encompass cells with essentially the same in vivo characteristics and functionality as primary hepatocytes (hepatocytes derived from primary cultures). Such characteristics can include one, or more of the following characteristics, e.g., cellular molecular markers such as the production of proteins such as albumin or alpha-1-antitrypsin or functionality e.g., enzyme production such as cytochrome P450 enzymes, Cyp3A4, Cyp3A4/Cyp3A7 ratio, glycogen storage or lipoprotein uptake, or visualization/mature markers such as bile canaliculi, absence of prominent vacuoles and LDL/glycogen storage, binucleation or the detection of FINF4α, (it is noted that HNF4α can be present in immature hepatocytes so it is not to be relied upon as a sole marker for mature highly functioning hepatocytes). Such molecular and functional characteristics of mature hepatocytes, and methods of testing for these characteristics to evaluate the suitability of hepatocytes for biological uses, are described. Such methods can include, for example, ELISA to detect protein secretion; mass spectrometry for enzyme activity, immunofluorescence and flow cytometry. Such assay methods are also known to those of skill in the art((see e.g., “High Efficient Differentiation of Functional Hepatocytes From Porcine Induced Pluripotent Stem Cells”, Ao et al. PLOS ONE vol. 9, issue 6, June 2014: 1-10; and “Efficient Generation of Functional Hepatocytes From Human Embryonic Stem Cells and Induced. Pluripotent Stem Cells by HNF4α Transduction” Takayama et al., Mol, Ther, 2012 Jan 20(1):127-137). Cells can also be evaluated for the expression of genes characteristic of mature hepatocytes using techniques well-known to those of skill in the art.

Culturing the iPSC under the conditions of metabolic stress as described herein (in the presence of metabolic stressors) results in an enriched population of purified or essentially/substantially purified, isolated hepatocytes, or mature, highly functional hepatocyte-like cells, suitable for therapeutic and diagnostic methods and other uses requiring functional, human hepatocytes, such as drug screening, medical devices, toxicity studies and organ transplant.

Thus, the present invention also encompasses isolated populations of cells (including purified, substantially purified or enriched populations when compared to iPSCs cultured under control conditions, i.e., not in the presence of metabolic stressors) produced from these methods and evaluated for hepatocyte characteristics as described above. Such an isolated cell population would comprise of at least about 10⁵, 10⁶, 10⁷; 10⁸, 10⁹, 10¹⁰ cells (or other number within the range of about 10⁵-10¹⁰ cells) and comprise at least about 90%-about 100% hepatocytes, or mature, highly functional hepatocyte-like cells. More specifically, the concentration of hepatocytes can range from about 80% to about 90%, 95%, 97%, 98%, and 99% and up to 100% hepatocytes in the cell population after suitable processing steps, e.g., flow cytometry. The isolated hepatocyte cells produced by the covered methods can be added to pharmaceutical solutions or diluents to form pharmaceutical compositions for use in the in vivo, or ex vivo treatment of liver diseases that would benefit from the use of hepatocytes to supplement non-functioning hepatocytes in a subject, such as a human. In particular, the isolated hepatocytes could be used to treat liver injuries or deficiencies, such as metabolic diseases, e.g., glycogen storage diseases or hyperlipidemias; degenerative diseases such as cirrhosis; or to treat injuries to the liver resulting from accidents or surgical procedures. Accordingly, the present invention also encompasses methods of treating liver diseases or injuries using the hepatocytes or mature, highly functional hepatocytes, produced by the methods described herein.

The culture conditions described herein for producing hepatocytes from cultured iPSC are unique and have notable advantages over the conditions currently employed by those skilled in the art, such as viral transduction. Therefore, another embodiment of the present invention encompasses the formulation of such culture media comprising components suitable for use as metabolic stressors, to be used at specific stages of growth and development of cultured cells to induce/direct the cells along the path of hepatocyte differentiation and function.

The methods described herein are particularly well-suited for scale-up for high throughput processes to obtain large quantities of the hepatocytes, or mature, highly functional hepatocyte-like cells.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

FIGS. 1A-B are schematics depicting stages of growth and development of cells that can be manipulated to promote enriched hepatocyte-like cell (HLC) populations. Enacting distinct metabolic stresses at different stages of lineage specification can result in hepatocyte-like cells.

FIG. 2 describes the key factors/components of protocols that can be used to induce hepatocyte specification at different stages of in vitro cultures.

FIG. 3 lists exemplary components of a Hepatocyte Selection Media (EISA,).

FIG. 4 describes an example of the metabolic pathway using galactose an alternative energy source.

FIG. 5A-D are graphs describing metabolic differences in fetal and adult liver tissue using assays for galactokinase 1 (GAL1),galactokinase 2 (GA2), ornithine transcarbamylase (OTC), phenyl alanine hydroxylase (PAH) and the housekeeping gene, RPL19.

FIG. 6 describes the key steps of fructose metabolism.

FIG. 7 is a schematic showing that the addition of ornithine and proline promote the glucogenic state.

### DETAILED DESCRIPTION OF THE PREFERRED EMBODIMENTS

Previous reports suggest that cells as early as hepatoblasts have the machinery to perform hepatic-specific metabolism. Previous results, however, using specific selection media were not conclusive in enriching for mature hepatocytes from iPSCs but selected out pluripotent cells. (Tomizawa et al., “Survival of Primary Human Hepatocytes and Death of Induced Pluripotent Stem Cells in Media Lacking Glucose and Arginine”, Plos One 2013) (Tomizawa et al., “Hepatoblast-like Cells Enriched from Mouse Embryonic Stem Cells in Medium Without Glucose, Pyruvate, Arginine, and Tyrosine”, Cell Tissue Res 2008.)

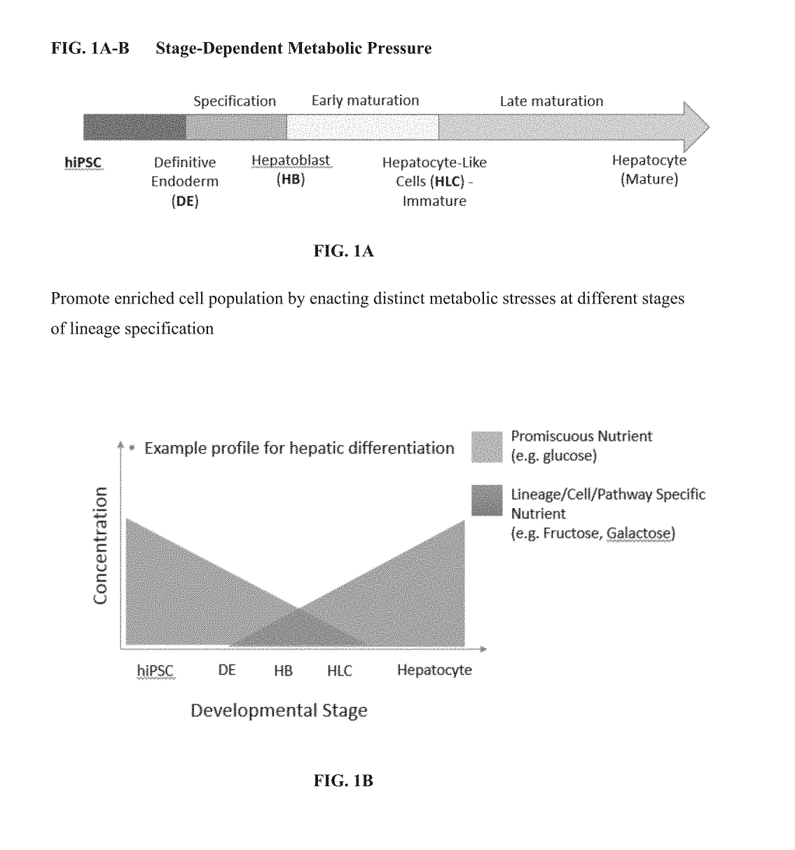

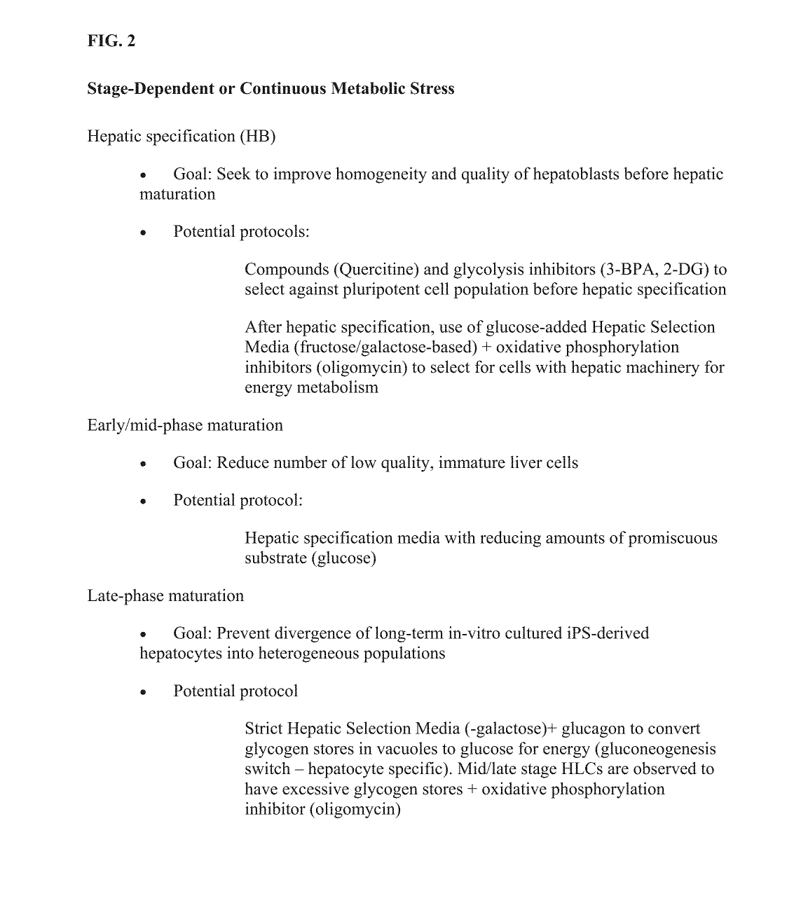

As described herein, developmental stage-dependent metabolic pressure can reasonably result in a population of cells that are enriched for hepatocytes, or mature, highly functional hepatocyte-like cells. (FIG. 1). In particular, as described in FIG. 2, during three stages of development of hepatocytes, small molecules and/or energy substrates in cell culture medium (e.g., Hepatic Cell Selection Media, or HSM) can direct the selection of cells that specifically use hepatic cell energy metabolism. Alternatively, metabolic stress can be exerted on cultured cells as constant stress or continuous stress to direct the selection of cells that specifically use hepatic cell energy metabolism rather than at specific time points during the culture protocol. For example, the method can induce hepatic specification at the hepatoblast stage before hepatic cell maturation. Compounds such as the anti-oxidant quercetin (also referred to as quercitine) and/or glycolysis inhibitors such as 3-BPA or 2-DG can be incorporated/added to cell culture media to select against the pluripotent cell population before hepatic cell specification.

Alternatively, after hepatic specification use of glucose-added Hepatic Selection Media, or HSM, (fructose/galactose based) plus oxidative phosphorylation inhibitors, such as oligotnycin, can be used to select for cells with hepatic machinery for energy metabolism.

Another stage of hepatic cell specification that can be targeted in the present method is the early/mid-phase maturation stage of hepatic cells. At this stage, the goal is to reduce the number of low quality, immature hepatic cells. In this method, amounts of promiscuous energy substrate, e.g., glucose, is reduced in the HSM.

Late-phase specification is another stage that can be targeted in the present method. The goal is to prevent divergence of long-term in vitro cultured iPSC-derived hepatocytes into heterogeneous populations. For example, the HSM is strict HSM-minus galactose and plus glucagon-to convert glycogen stores in cell vacuoles to glucose for energy. This glucogneonesis switch is hepatocyte specific.

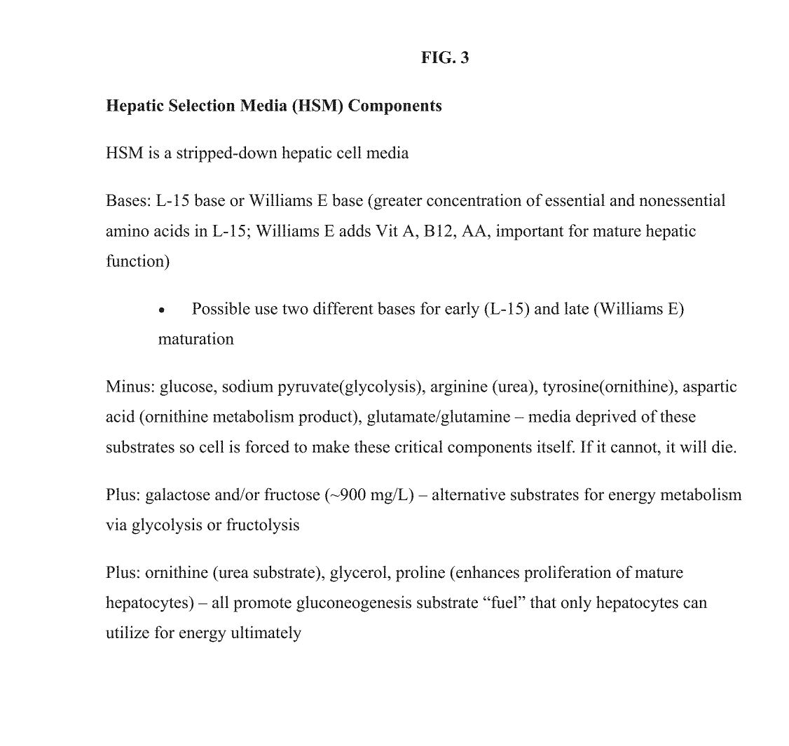

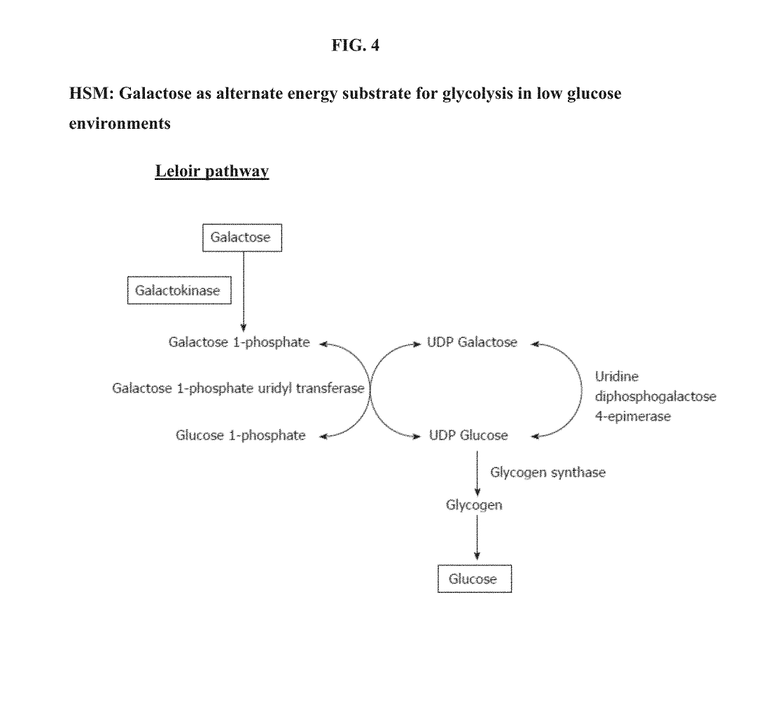

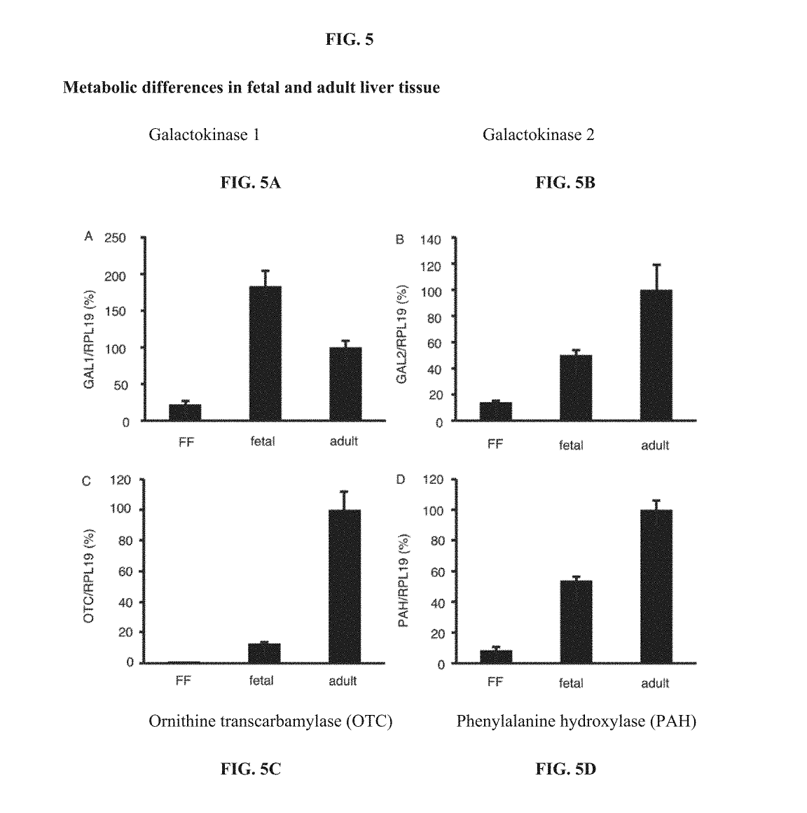

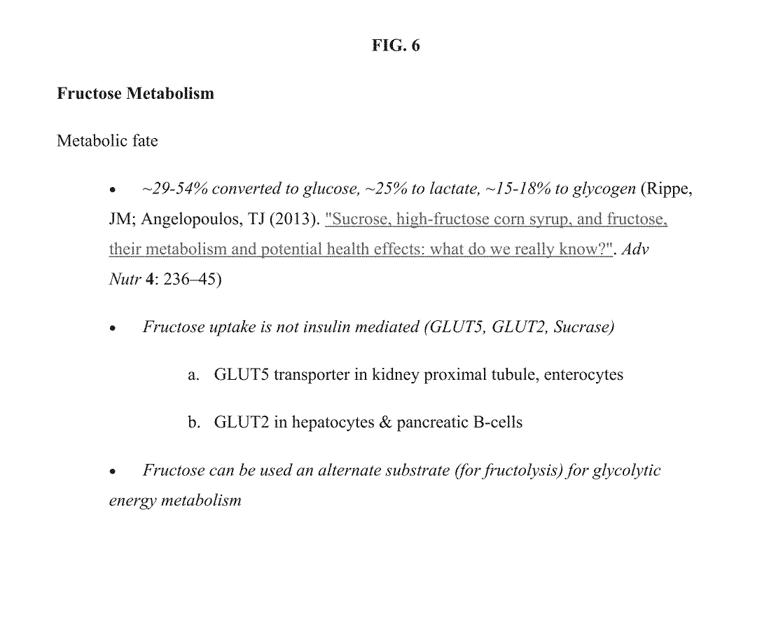

As described in FIG. 3, the present invention encompasses a number of hepatic cell selection media (HSM) suitable for use in methods of producing hepatocytes from induced pluripotent stem cells. In particular, the HSMs described herein can be used to culture iPSC to direct the differentiation of i.PSC by introducing or inducing metabolic stress through early-stage, mid-stage and late-stage maturation into functional hepatocytes by varying the type and concentration of energy substrates in the media (FIGS. 4-7), and/or contacting the cells with, or introducing into the cell culture medium, components, drugs or molecules, in suitable concentrations and culture conditions, to inhibit specific metabolic pathways e.g., oxidative phosphorylation and force cells to use alternate energy sources for survival,

Hepatic Selection Media (HSM) is a stripped down hepatic cell media. The base of the media can be for example, L-15 base, or Williams E base. L-15 has a greater concentration of essential and non-essential amino acids. The Williams E adds vitamins A, B12, and amino acids important for mature hepatocytes. The suitability of either media base can depend on the stage of maturation that is being targeted. For example, targeting an early stage of maturation is can be more suitable to use L-15. If a later stage is targeted, then Williams-E can be the more suitable media base.

HSM lacks glucose, sodium pyruvate (for glycolysis), arginine (in the urea cycle), tyrosine (ornithine) aspartic acid (omithine metabolism product) and glutamate/glutamine. Media deprived of these components results in forcing/directing the cells to produce these critical components for its survival.

HSM contains galactose and/or fructose or other energy source (for example, at a. concentration of about 900 mg/L) which act as alternate substrates for energy metabolism via glycolysis or fructolysis. Additionally other components can be added to HSM such as ornithine (a urea substrate), glycerol, and/or proline (to enhance proliferation of mature hepatocytes). These components al promote gluconeogenesis substrates to act as fuel that only hepatocytes can ultimately utilize for energy.

FIG. 4 shows an energy producing pathway, the Leloir pathway, whereby hepatocytes can use galactose as an alternate energy substrate for glycolysis in low glucose environments. As shown in the pathway diagram, galactokinase is specific to hepatocyte lineage cells (e.g., hepatoblasts, etc.) and is required to convert galactose to glucose. Galactose can then become a substrate for glycolytic energy production if low amounts of glucose are present in the cell culture media. As the results of experiments shown in FIG. SA-D, galactokinases exist in fetal and adult liver tissue, and ornithine transcarbamylase (OTC) and phenylalanine hydroxylase (PAH) are significantly higher in mature/adult liver tissue because mature liver cells are more dependent on urea-assisted metabolism (e.g., using OTC).

Fructose can also be used as an alternate energy substrate (for fructolysis) for glycolytic energy metabolism. (See e.g., ˜29-54% converted to glucose, ˜25% to lactate, ˜15-18% to glycogen (Rippe, J M; Angelopoulos, T J (2013), “Sucrose, high-fructose corn syrup, and fructose, their metabolism and potential health effects: what do we really know?” Adv. Nutr 4: 236-45). Fructose uptake is not insulin mediated (GLUT5, GLUT2, Sucrase), The GLUT5 transporter is found in the kidney proximal tubule and enterocytes, whereas the GLUT2 transporter is found in hepatocytes & pancreatic B-cells.

The HSM as described herein can be arginine depleted. Arginine depletion forces production of endogenous arginine through the urea cycle in the liver. Arginine is typically added to media because it is not made by most cells. However, arginine can be made during the urea cycle, a pathway specific only to hepatocytes. Therefore, media lacking arginine can place metabolic stress on cells that cannot synthesize new arginine. Additionally, the addition of ornithine can promote expedited urea cycle and arginine production.

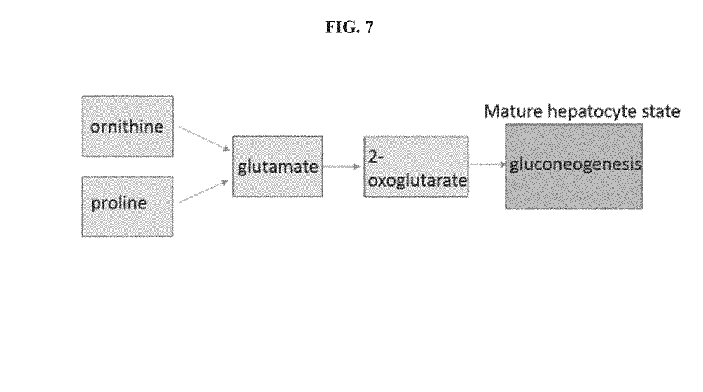

The HMS as described herein can comprise components such as ornithine and proline to promote the gluconeogenic state and urea cycle (see FIG. 7). Glutamate from each catabolic step can be used to make 2-oxyglutarate which is precursor for gluconeogenesis and endogenous glucose production. The addition of glucagon to the HSM forces glycogen conversion to glucose. For example, immature hepatocyte-like cells display robust vacuoles of excess glycogen storage and depleting these stores can remove potential roadblocks toward maturity.

The HSM as described herein can also be depleted of tyrosine. The presence of tyrosine promotes hydroxylation of phenylalanine, which occurs exclusively in kidney and liver. The lack of tyrosine supplementation leads to accumulation of phenylalanine and depletion of 2-oxoglutarate, a substrate for the TCA cycle and oxidative phosphorylation energy metabolism. This switches energy metabolism form oxidative phosphorylation, hick all differentiated cells prefer, toward glycolytic metabolism, which, in a low glucose environment with alternate substrates (e.g., galactose and/or fructose) will kill off non-hepatic cells.

Alternatively, switching energy programs can be done using small molecule metabolic inhibitors. Some suitable inhibitors can be, for example, 3-bromopyruvic acid or 2-deoxy-d-glucose (2-DG). The use of these inhibitors select out pluripotent stem cells that only use glycolysis for energy. For example, in one embodiment, inhibiting glycolysislglucose metabolism using 3-brotnopyruvic acid inhibits mitochondria-bound IIK2 which metabolizes glucose for glycolysis. (See for example, Marache et al. Chem. Sci. 2015; Gong, et al. :3-bromopyruvic acid, A Hexokinase II Inhibitor, is an Effective Antitumor Agent on the Hepatoma Cells” Anti-cancer Agents in Med. Chem.; vol. 14, issue 5, 2014). 2-DG can also be used to inhibit glucose metabolism by inhibiting fructose-6-phosphate production and anaerobic glycolysis.

Another example of a suitable small molecule inhibitor to use is oligomycin A, which inhibits oxidative phosphorylation. More specifically, oligomycin A inhibits ATP synthesis (the H) portion) and re-routes energy production to glucose-based anaerobic metabolism. Switching energy production to glycolysis, in conjunction with HMS as described herein, can select out cells that cannot make energy from liver-specific program, in particular at the hepatoblast specification stage and with maturing iPS-hepatocytes.

Using suitable and specific FI,SM throughout (continuously), or at different stages (stage-dependent) of the growth and development of cultured iPSC results in mature, fully functional hepatocytes. The different stages of development that can be targeted for the inducement of metabolic stress by culture conditions include the initial culture conditions, through the definitive endoderm cell stage, through early maturation of the hepatoblasts to immature hepatocyte-like cells and finally to mature hepatocytes or mature, highly functional hepatocyte-like cells, resulting in a population of cells enriched for, or of purified cells or isolated cells with an in vivo-like liver phenotype. Evaluating the cells produced by the methods described herein can be done by metabolic and visual assays that are known to those of skill in the art.

For example, protein secretion assays such as ELISAs can be used to identify/detect cells with mature hepatocyte phenotypes (e.g., mature, highly functioning hepatocyte-like cells). More specifically the assay can be for biological markers characteristic of the stage of hepatocyte development. Albumin can be one of those markers, whereas albumin is found in higher concentrations/levels for the mature hepatocyte phenotype (>6 μg/day/106 cells). Alpha-fetoprotein is another marker, whereas alpha-fetoprotein is found in lower levels for the mature phenotype (<100 ng/day/106 cells).

Enzyme activity assays can also be used to identify the developmental stage/phenotype of the hepatic cell, and can include for example, substrate assays and mass spectroscopy. Cyp3A4 has higher activity for the mature phenotype (>1.5 pmol/min/106 cells), whereas Cyp3A4/Cyp3A7 demonstrates lower activity for the mature phenotype (<5 pmol/min/106 cells).

Visualization of cells and detection of mature markers using, for example, immunofluorescence can also be used to identify the phenotype of the hepatic cell. For example, the characterization of the bile canaliculi; absence of prominent vacuoles and LDL/glycogen storage or binucleation of the cell/cell nucleus can be useful as markers to determine the phonotype of the cell. Markers such as HNF4α, Cyp3a4, αlαT can be detected using flow cytometry. Finally, gene expression can also be used to determine the phenotype of the cell. (See, for example, Wang, X J; et. al., “Relationship between hepatic phonotype and changes in gene expression in cytochrome P450 reductase (POR) null mice”, Biochem. J., vol. 3: issue 3, Jun. 2005); Gatti, D. et. al. “Genome-level analysis of genetic regulation of liver gene expression networks”, Hepatology, 2007 August; 46(2):548-557)).

Therefore, as described herein, hepatocytes exhibit unique metabolic characteristics, which can be exploited in cell culture to produce hepatocytes, or mature, functional hepatocyte-like cells from iPSC. Media lacking specific substrates or comprising small molecules that inhibit certain metabolic pathways, force cultured cells to either differentiate into mature hepatocytes or die. It is reasonable to believe that by modifying metabolic pathways specific for hepatocytes during cell culture will result in a population of functional hepatocytes or mature, highly functional hepatocyte-like cells suitable for transplant, therapeutic and diagnostic uses.

Exemplary Protocol for Obtaining Hepatocytes from iPSC:

An exemplary method of generating hepatocytes, or mature, highly functioning hepatocyte-like cells from induced pluripotent stern cells (iPSC) comprises the following steps. Those skilled in the art understand that these steps can be modified, or slightly altered from the conditions described below and still successfully obtain the desired hepatocytes, or mature, highly functioning hepatocyte-like cells. The induced pluripotent mammalian stem cells, specifically human iPSC, are initially cultured under conditions sufficient to maintain cell growth, thereby providing an iPSC cell culture.

The culture conditions are then modified to improve homogenecity and quality of hepatoblasts before hepatic maturation and to inhibit glycolysis by incorporating one, or more of components into the culture media for a time sufficient to inhibit glycolysis, e.g., about 24 to about 96 hours. Glycolysis inhibitors such as quercitine in a dose range (i.e., concentration range) of about 1 to about 10 μM for about 1 to about 3 days duration; 3-bromopyruvic acid in a dose range of about 10 to about 25 for about 2- to about 4 days; or 2-deoxy-d-g;ucose in a dose range of about 10 to about 50 mM for about 24 hours can be used. Inhibition of glycolysis selects against the iPSC in culture and promotes enrichment of the hepatoblasts in the culture.

The population of cells enriched for hepatoblasts are recovered and then cultured in the presence of suitable Hepatic Cell Selection Media (HSM) and oxidative phosphorylation inhibitors to promote early maturation of the population of hepatoblast cells, directing the hepatoblasts toward hepatic specification. The culture media incorporates, for example, Oligomycin A in a dose range of about 1 to about 10 μM for about 24 to about 96 hours to select for immature hepatic cells (cells with hepatic cell characteristics).

The immature hepatic cells are then recovered and cultured in the presence of a fructose and/or galactose-based HSM while reducing the amounts of glucose in the media to select for cells with specific hepatic machinery for energy metabolism. For example, the galactose or fructose concentrations in the culture media at this step can be about 5 g/l for up to about 10 days.

The final step of the method comprises recovering the population of immature hepatic cells and culturing the cells in the presence of suitable IBM to promote gluconeogenesis in the cells and to prevent divergence of long-term, in vitro iPSC-derived hepatocytes into heterogeneous populations In the mid/late stage of development, hepatic like cells are observed to have excessive glycogen store. For example, strict hepatic selection media can comprise glucagon but lack galactose to promote conversion of glycogen stores in cellular vacuoles to glucose for energy (a gluconeogenesis switch that is hepatic cell specific). The HSM can also include an oxidative phosphorylation inhibitor such as oligomycin as described above. This final step results in an enriched population of hepatocytes and/or mature, highly functioning hepatocyte-like cells.

All references cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of producing hepatocytes, or mature hepatocyte-like cells from induced pluripotent stem cells, comprising the steps of: culturing the induced pluripotent stem cells under conditions suitable for inducing metabolic stress at different stages of hepatic lineage specification which induces the stem cells to differentiate into hepatocytes or mature highly functional hepatocyte-like cells, thereby producing hepatocytes, or mature highly functional hepatocyte-like cells.

2. The method of claim 1 wherein the induced pluripotent stem cells are human cells.

3. A hepatocyte, or mature, highly functional hepatocyte-like cell produced by the method of claim 1.

4. The method of claim 1 wherein the metabolic stress is continuous throughout the cell culture conditions.

5. The method of claim 1, wherein the metabolic stress is induced during cell culture at the hepatoblast lineage specification stage.

6. The method of claim 1, wherein the metabolic stress is induced during cell culture at the early/mid-phase of the hepatic lineage specification stage.

7. The method of claim 1, wherein the metabolic stress is induced during cell culture at the late-phase of the hepatic lineage specification stage.

8. A culture medium for differentiating induced pluripotent stern cells into hepatocytes, or mature, highly functional hepatocyte-like cells comprising glycolysis inhibitors.

9. A culture medium for differentiating induced pluripotent stern cells into hepatocytes, or mature hepatocyte-like cells comprising oxidative phosphorylation inhibitors.

10. A culture medium for differentiating induced pluripotent stem cells into hepatocytes, or mature hepatocyte-like cells comprising metabolic substrates at concentrations suitable for inducing hepatic-specific metabolic processes for energy production.

11. A composition comprising a cell population of at least 90% hepatocytes, or mature, highly functional hepatocyte-like cells and a culture medium suitable for maintaining hepatocytes, or mature hepatocyte-like cells, wherein the hepatocytes, or mature hepatocyte-like cells are produced by the method of claim 1.

12. A method of generating hepatocytes, or mature, highly functioning hepatocyte-like cells from induced pluripotent stem cells (iPSC), the method comprising the steps of:
a.) Providing an iPSC culture;
b.) Inhibiting glycolysis in the culture to select for hepatoblast cells during the hepatic specification stage;
c.) Recovering the hepatoblasts of step b.) and culturing the hepatoblasts in the presence of suitable Hepatic Cell Selection Media (HSM) and oxidative phosphorylation inhibitors to select for immature hepatic cells;
d.) Recovering the immature hepatic cells of step c.) and culturing the cells in the presence of suitable HSM while reducing the amounts of glucose in the media to reduce the population of immature hepatic cells;
e.) Recovering the cells of step d.) and culturing the cells in the presence of suitable HSM to promote gluconeogenesis in the cells, resulting in a population of hepatocytes or mature, highly functioning hepatocyte-like cells.

13. The method of claim 12, step b.) wherein the culture media comprises a glycolysis inhibitor selected from the group consisting of: quercitine, 3-bromopyruvic acid or 2-deoxy-D-glucose.

14. The method of claim 12, step c.), wherein the culture media comprises oligomycin A.

15. The method of claim 12, step d.), wherein the media comprises galactose, fructose or a combination of the two.

16. The method of claim 12, step e.), wherein the culture media comprises glucagon.

17. The method of claim 12, step b.), where the glycolysis inhibitor is quercitine in a dose range of about 1 to 10 for about 1 to 3 days duration.

18. The method of claim 12, step b.), wherein the glycolysis inhibitor is 3-bromopyruvic acid in a dose range of about 10-25 for about 2-4 days.

19. The method of claim 12, step b.), wherein the glycolysis inhibitor is 2-deoxy-d-glucose in a dose range of about 10-50 mM, for about 24 hours.

20. The method of claim 12, step c.), wherein the oxidative phosphorylation inhibitor is Oligotnycin A in a dose range of about 1-10 μM, for about 24-96 hours.

21. The method of claim 12, step d.), wherein the galactose or fructose concentration is about 5 g/l, for up to 10 days.

22. The method of claim 12, step d.), wherein the HSM can also include an oxidative phosphorylation inhibitor selected form the group consisting of: oligomycin, rotenone or rutamycin.
